(19) United States
(12) Patent Application Publication
(10) Pub. No.: US 2025/0316025 A1
(43) Pub. Date: Oct. 9, 2025

(54) SYSTEMS AND METHODS FOR MOTION-ROBUST 3D RECONSTRUCTION AND MEASUREMENT OF BODY PARTS

(71) Applicant: PediaMetrix Inc., Rockville, MD (US)

(72) Inventors: Can KOCABALKANLI, Rockville, MD (US); Reza SEIFABADI, North Potomac, MD (US); Fereshteh AALAMIFAR, North Potomac, MD (US); Yifan YIN, Baltimore, MD (US); Mingxu LIU, Baltimore, MD (US); A N M Tawsifur RAHMAN, Baltimore, MD (US); Bo WU, Rockville, MD (US)

(73) Assignee: PediaMetrix Inc., Rockville, MD (US)

(21) Appl. No.: 19/174,488

(22) Filed: Apr. 9, 2025

Related U.S. Application Data
(60) Provisional application No. 63/631,776, filed on Apr. 9, 2024.

(51) Int. Cl. G06T 17/20; G06T 5/73; G06T 7/00; G06T 7/194; G06T 7/70; G06V 10/74

(52) U.S. Cl. CPC G06T 17/20; G06T 5/73; G06T 7/0012; G06T 7/194; G06T 7/70; G06V 10/74; G06T 2210/56

(57) ABSTRACT

A system, including an image sensor configured to acquire a plurality of images of a body part of a patient; and processing circuitry configured to receive the images of the body part of the patient and depth data including depth values corresponding to pixels of each image of the body part of the patient, isolate a foreground region of interest including the body part of the patient in the plurality of images, determine image sensor poses in three-dimensional space based on depth data, the depth data including depth values corresponding to pixels of each image of the body part of the patient, generate a combined point cloud of pixels corresponding to the foreground region of interest based on the depth data and the image sensor poses, and generate a mesh surface of the body part of the patient.

### CROSS-REFERENCE TO RELATED APPLICATIONS

The present application claims priority to U.S. Provisional Application No. 63/631,776, filed Apr. 9, 2024 which is hereby incorporated by reference in its entirety for all purposes.

### GOVERNMENT LICENSE RIGHTS

This invention was made with government support under Grant Number R44 DE031461 awarded by the National Institutes of Health. The government has certain rights in the invention.

### BACKGROUND

### Field of the Disclosure

The present disclosure relates to systems and methods for generating an accurate and robust three-dimensional (3D) model of the body, e.g., head, ear, spine, limbs, torso, teeth, for anthropometric measurements and diagnosis of abnormalities and in order to monitor and track growth in a convenient manner.

### Description of the Related Art

About 47% of infants between 7-12 weeks develop some form of head deformities. There are two types of head deformities: synostotic and non-synostotic. The former involves pre-mature fusion of skull sutures and can be morbid often requiring surgery. The more common type is non-synostotic which includes deformational plagiocephaly and brachycephaly, or DPB. In addition to cosmetic effects and the associated psychological pressure, several studies have shown DPB is related to a series of developmental delays that may continue through 3 years of age. Usually, there is little awareness about head deformity conditions risks especially among first time parents. Currently, pediatricians do not routinely perform a quantitative method to measure head shape. In the case of head deformity, the timing of the diagnosis is an important factor in determining the treatment method. It is also important to detect head deformations early to initiate timely therapy, such as surgery, conservative therapy or helmet therapy.

The foregoing “Background” description is for the purpose of generally presenting the context of the disclosure. Work of the inventors, to the extent it is described in this background section, as well as aspects of the description which may not otherwise qualify as prior art at the time of filing, are neither expressly or impliedly admitted as prior art against the present invention.

### SUMMARY

The present disclosure relates to systems and methods of measuring the body based on image analysis.

According to an embodiment, the present disclosure relates to a system, comprising: an image sensor configured to acquire a plurality of images of a body part of a patient; and processing circuitry configured to receive the images of the body part of the patient, isolate a foreground region of interest including the body part of the patient in the plurality of images, determine image sensor poses in three-dimensional space based on depth data, the depth data including depth values corresponding to pixels of each image of the body part of the patient, generate a combined point cloud of pixels corresponding to the foreground region of interest based on the depth data and the image sensor poses, and generate a mesh surface of the head of the patient based on the combined point cloud.

According to one embodiment, the present disclosure relates to a non-transitory computer-readable storage medium for storing computer readable instructions that, when executed by a computer, cause the computer to perform a method, the method, comprising: receiving a plurality of images of a body part of a patient and depth data including depth values corresponding to pixels of each image of the body part of the patient; isolating a foreground region of interest including the body part of the patient in the plurality of images; determining image sensor poses in three-dimensional space based on the depth data; generating a combined point cloud of pixels corresponding to the foreground region of interest based on the depth data and the image sensor poses; and generating a mesh surface of the body part of the patient based on the combined point cloud.

According to one embodiment, the present disclosure relates to a method, comprising: receiving, via processing circuitry, a plurality of images of a body part of a patient and depth data including depth values corresponding to pixels of each image of the body part of the patient; isolating, via the processing circuitry, a foreground region of interest including the body part of the patient in the plurality of images; determining, via the processing circuitry, image sensor poses in three-dimensional space based on the depth data; generating, via the processing circuitry, a combined point cloud of pixels corresponding to the foreground region of interest based on the depth data and the image sensor poses; and generating, via the processing circuitry, a mesh surface of the body part of the patient based on the combined point cloud.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is an illustration of types of craniosynostosis, according to one embodiment of the present disclosure;

FIG. 2 is method of determining cranial parameters, according to one embodiment of the present disclosure;

FIG. 3A is an illustration of a user device, according to one embodiment of the present disclosure;

FIG. 3B is an illustration of a user device, according to one embodiment of the present disclosure;

FIG. 4A is an illustration of a scan acquisition when the subject is stationary, according to one embodiment of the present disclosure;

FIG. 4B is an illustration of a scan acquisition when the subject is stationary, according to one embodiment of the present disclosure;

FIG. 4C is an illustration of a scan acquisition when the subject is stationary, according to one embodiment of the present disclosure;

FIG. 5 is an illustration of a scan acquisition when the subject rotates, according to one embodiment of the present disclosure;

FIG. 6A is an illustration of a scan acquisition user interface, according to one embodiment of the present disclosure;

FIG. 6B is an illustration of a scan acquisition user interface, according to one embodiment of the present disclosure;

FIG. 6C is an illustration of a scan acquisition user interface, according to one embodiment of the present disclosure;

FIG. 7A is an illustration of a scan cap, according to one embodiment of the present disclosure;

FIG. 7B is an illustration of a scan cap, according to one embodiment of the present disclosure;

FIG. 7C is an illustration of a scan cap, according to one embodiment of the present disclosure;

FIG. 8 is an illustration of foreground estimation, according to one embodiment of the present disclosure;

FIG. 9A is an illustration of a depth map, according to one embodiment of the present disclosure;

FIG. 9B is an illustration of a depth map, according to one embodiment of the present disclosure;

FIG. 10A is an illustration of a closed loop, according to one embodiment of the present disclosure;

FIG. 10B is an illustration of a loop closure algorithm, according to one embodiment of the present disclosure;

FIG. 11A is an illustration of surface area coverage, according to one embodiment of the present disclosure;

FIG. 11B is an illustration of surface area coverage in a top view, according to one embodiment of the present disclosure;

FIG. 12A is an illustration of scan acquisition with a first exposure time, according to one embodiment of the present disclosure;

FIG. 12B is an illustration of scan acquisition with a second exposure time, according to one embodiment of the present disclosure;

FIG. 13 is an illustration of scan acquisition with different frames per second, according to one embodiment of the present disclosure;

FIG. 14A is an illustration of scan acquisition with a first frames per second, according to one embodiment of the present disclosure;

FIG. 14B is an illustration of scan acquisition with a second frames per second, according to one embodiment of the present disclosure;

FIG. 15 is an illustration of feature matching, according to one embodiment of the present disclosure;

FIG. 16 is an illustration of camera pose determination, according to one embodiment of the present disclosure;

FIG. 17A is an illustration of a mesh surface with texture, according to one embodiment of the present disclosure;

FIG. 17B is an illustration of a mesh surface without texture, according to one embodiment of the present disclosure;

FIG. 18A is an illustration of nasion landmark detection, according to one embodiment of the present disclosure;

FIG. 18B is an illustration of tragion landmark detection, according to one embodiment of the present disclosure;

FIG. 19 is an illustration of landmark detection, according to one embodiment of the present disclosure;

FIG. 20A is an illustration of landmark detection on a mesh in a front view, according to one embodiment of the present disclosure;

FIG. 20B is an illustration of landmark detection on a mesh in a side view, according to one embodiment of the present disclosure;

FIG. 21 is an illustration of a mesh coordinate system, according to one embodiment of the present disclosure;

FIG. 22A is an illustration of a head contour polygon, according to one embodiment of the present disclosure;

FIG. 22B is an illustration of a head contour, according to one embodiment of the present disclosure;

FIG. 23 is an illustration of principal component projections, according to one embodiment of the present disclosure;

FIG. 24 is an illustration of a cranial report, according to one embodiment of the present disclosure;

FIG. 25 is an illustration of cranial measurement accuracy, according to one embodiment of the present disclosure; and

FIG. 26 is a hardware description of a mobile device, according to one embodiment of the present disclosure.

### DETAILED DESCRIPTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

Head deformities can be synostotic, caused by the premature ossification and closing of sutures in the skull, or non-synostotic, caused by other, external factors. There are three main types of non-synostotic head deformity. Plagiocephaly refers to cases where the infant's head has developed asymmetrically and there is flatness in one side of the head and bulging in the other side. Brachycephaly describes cases where the back of the head is flat, i.e., the head is wider than normal. Finally, scaphocephaly is the term used when the head is longer than normal. For each type, a severity score can be determined: mild, moderate, or severe. In addition, an infant might have a combination of plagiocephaly and brachycephaly, also called asymmetrical brachycephaly. There are two most common methods of treatment of head deformity. The first approach, which is recommended in early stages, is called repositioning therapy and can be done by parents at home. Repositioning therapy involves positioning the baby's head such that pressure is placed at the bulging area while enough room for growth is left at the flat side. For example, the baby can be placed on their stomach while awake; however, many babies do not favor this position, and this approach does not really address what is causing the flattened head. Placing the baby on their side or on their back looking sideways, as well as using special pillows with cavities, are other methods that can be practiced.

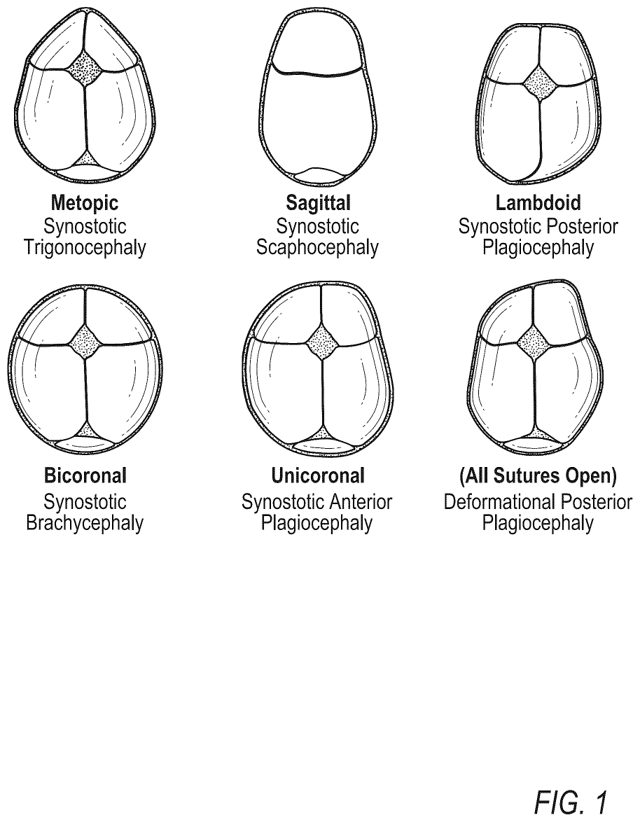

If the head shape is still deformed after trying repositioning therapy or if head deformity is discovered too late, helmet therapy becomes the most viable option. In helmet therapy, the infant's head shape is measured in three dimensions (using a 3D scanner) and a customized correctional helmet is built. This helmet should ideally be worn by the infant 23 hours a day for several weeks and up to a few months. Unfortunately, the helmet is inconvenient, expensive, and not reimbursable by many insurance plans, while repositioning therapy is most effective if it is applied when the baby's head growth is significant (usually before 4-6 months of age). Another common condition that induces head malformations in children is craniosynostosis, which is caused by the premature fusion of one or more of the head sutures. Unlike non-synostotic head deformities, craniosynostosis cannot benefit from conservative therapy options such as repositioning or helmet therapy, and may require surgical intervention between 3-6 months of age as it can impact the patient's cerebral development and vision if not treated in time. FIG. 1 is an illustration of types of craniosynostosis.

Diagnosis of non-synostotic head deformity conditions can be made by measuring the head shape using a special caliper called craniometer. Synostotic cases are diagnosed by specialists such as pediatric neurosurgeons and plastic surgeons. Parents are referred by their infant's pediatrician to specialists, such as orthotists or pediatric neurosurgeons, to evaluate the type and severity of head deformity. The waiting times for these appointments may be up to a month or more, depending on the geographical location. The parameters measured during the head shape evaluation appointment include cephalic index (CI) and cranial vault asymmetry index (CVAI), where CI refers to head width to length ratio and CVAI refers to the ratio of asymmetry between right and left diagonals of head. These parameters can also be calculated using a two-dimensional (2D) top view image of the baby's head with equivalent diagnostic accuracy. It should be noted that these indices or a visual alone may not be sufficient to distinguish non-synostotic deformities from most types of craniosynostosis in a 2D image, and this diagnosis usually has to be done at a craniofacial clinic, which may need to use a computerized tomography (CT) scan or similar method to examine the head sutures.

In one embodiment, the present disclosure is directed to automated systems and methods that can be used to scan an infant's body part (e.g. the head) and that can automatically measure and analyze the shape of the body part and can diagnose deformities such as synostotic and non-synostotic head deformities. The automated systems and methods can help with earlier detection and diagnosis of these conditions, allowing the most appropriate and least invasive treatment option available to be pursued. In one embodiment, the systems and methods of the present disclosure can include determining information such as cranial volume, which can be an important developmental metric for a baby's skull. In one embodiment, the 3D models of the body can be useful in identifying other deformities such as those occurring in the ear, or spine (e.g. scoliosis), teeth, limbs, etc.

In one embodiment, the present disclosure is directed to generating and analyzing a 3D model of a baby's head in order to diagnose and differentiate between synostotic and non-synostotic deformities. Analyzing a 3D model of a baby's head can be helpful and can provide information not available through visual or 2D image-based analysis, without the need for a CT or similar 3D imaging methods that are harmful for infants due to radiation risks. Furthermore, 3D models according to the present disclosure can be reconstructed from imaging data that can be collected from more convenient hardware options, such as a user device having a camera (e.g. a mobile device) and/or attachable depth sensors or certain smartphones with embedded depth sensors such as an iPhone

Traditional 3D reconstruction methods may fail when a subject moves during imaging. Infants who are in a vulnerable age range for head deformities often move during imaging and thereby prevent accurate 3D reconstruction based on the acquired images. Since 3D reconstruction methods may use multiple images from different angles to reconstruct a complete representation of the head, subject movement makes it difficult, if not impossible, to ensure that the necessary images are obtained or to infer the positional relationships of acquired images in order to combine images to create a 3D image (also referred to as a 3D model). Using more than one camera to acquire the multiple images can be bulky, expensive, require frequent calibrations, inconvenient, or inaccessible due to the cost. Using patterns or markers with known locations on the head or facial landmarks to make it easier to estimate the camera's pose and combine information from multiple frames, may be cumbersome and ineffective. For example, using markers with known locations may require a user to successfully place markers precisely at the intended locations or may require a specific design, and facial landmarks may not be visible from different camera position. Furthermore, many medical 3D reconstruction systems require manual annotation of landmarks by an analyst or specialist to complete measurements.

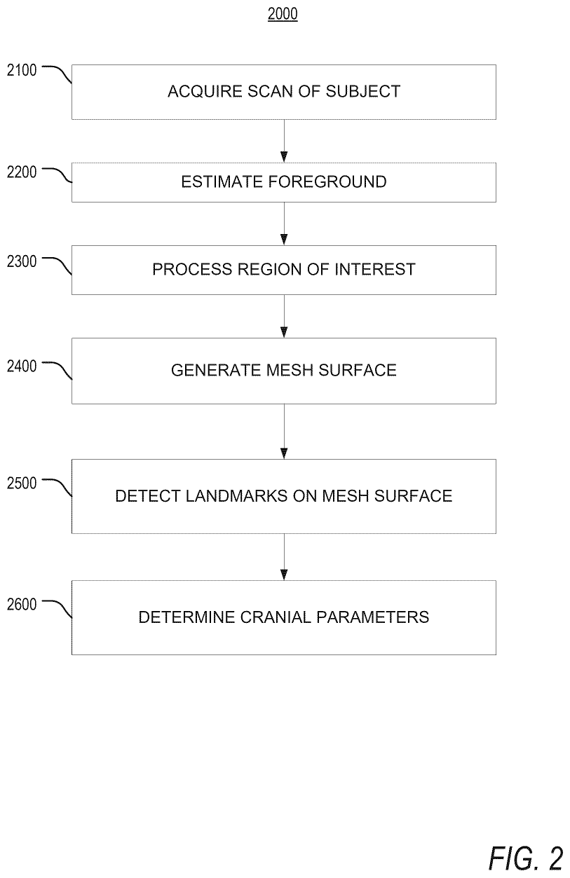

In one embodiment, the present disclosure is directed to systems and methods for 3D reconstruction through foreground estimation and usage of depth data that are tolerant of subject movement and that do not require placement of markers at pre-determined locations. FIG. 2 is a flowchart of a method 2000 of 3D reconstruction according to an embodiment of the present disclosure. As an example, the method 2000 can be used to reconstruct and analyze head shape in order to identify deformities. In step 2100, a scan can be acquired of a subject. The scan can include one or more images. In step 2200, foreground estimation can be applied to identify and segment a region of interest throughout the scan, e.g. a body part of interest such as the head. In step 2300, the scan can be analyzed and/or processed to improve the image quality of the region of interest and/or the usefulness of the region of interest for a 3D reconstruction. The processing of step 2300 can include one or more types of processing including motion compensation. In step 2400, a mesh surface of the region of interest can be generated as a 3D representation using mesh generation techniques. In step 2500, landmarks on the region of interest of the mesh surface can be automatically (or manually) detected based on 2D and/or 3D space. In step 2600, cranial parameters can be determined based on the mesh surface and the landmarks.

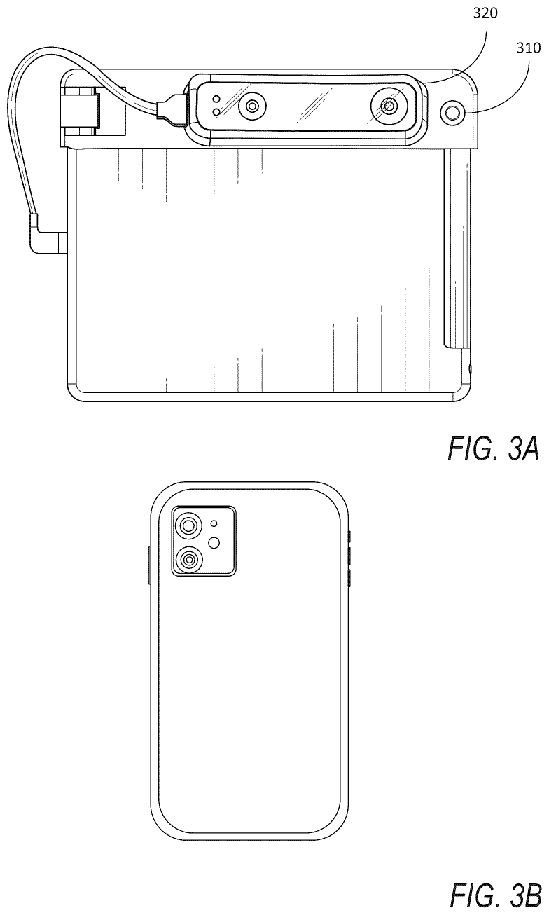

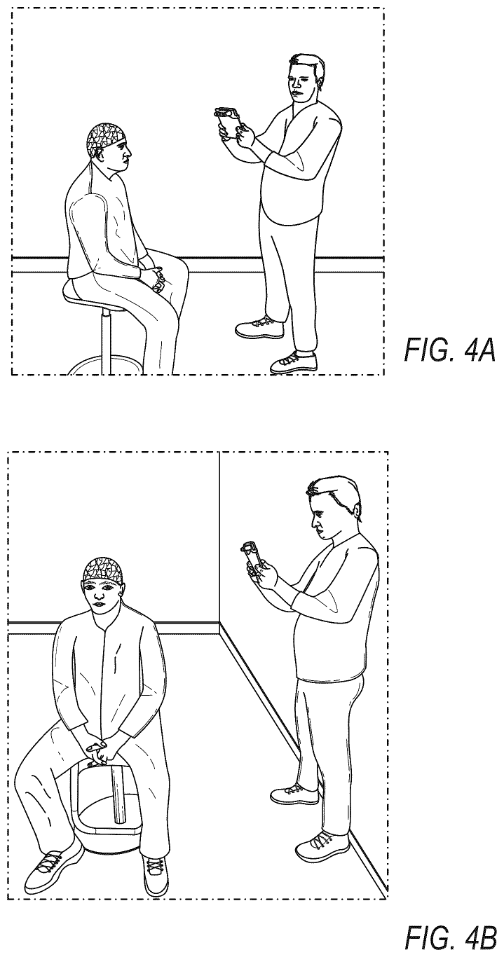

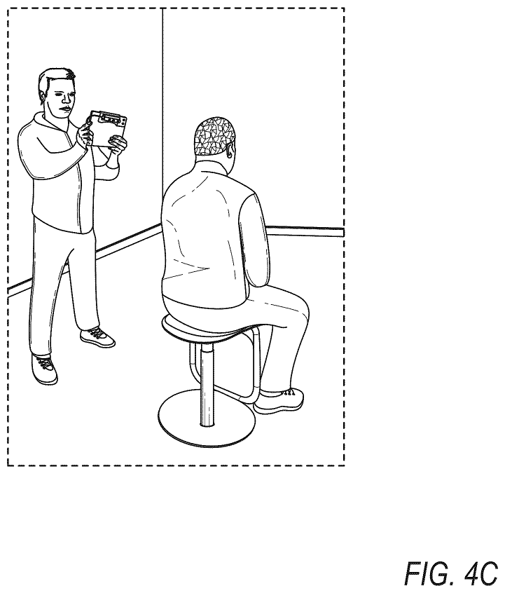

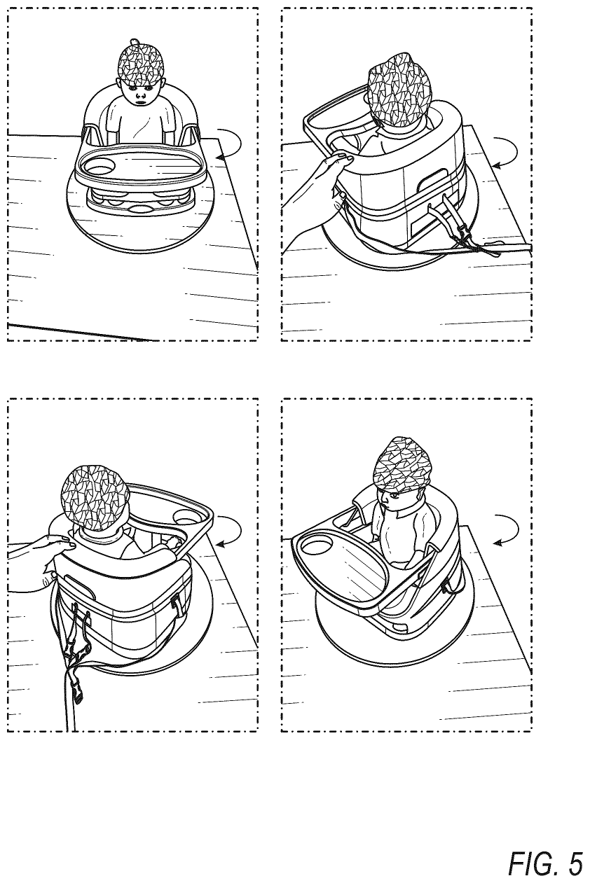

In one embodiment, the scan acquisition can include acquiring scan data including one or more images (frames) of a subject using a camera. The camera can include depth sensors or can be coupled with depth sensors. FIG. 3A is an illustration of a user device, e.g. a tablet, having a camera 310 and depth sensor hardware 320. In one embodiment, the camera 310 and the depth sensor hardware 320 can be coupled to a mobile device. As an example, a depth sensor can be a light detection and ranging (LIDAR) sensor, or structured light sensor FIG. 3B is an illustration of a user device, e.g., a smartphone, having an embedded camera and depth sensor. The acquired scan data can include depth data. For example, the acquired scan data can include a LIDAR or structured light point cloud or depth map. In one embodiment, the scan acquisition can include determining depth data based on image data that is acquired by the camera. Depth data can be determined photogrammetrically using two-dimensional image data, e.g., red-green-blue (RGB) image data. Photogrammetric determination of depth data can include identifying common points or features between two sequential images and determining a depth of the features based on displacement of the features between images. FIGS. 4A through 4C are illustrations of a scan acquisition process when the subject is stationary and the camera moves around the subject. In one embodiment, the scan can be an approximately 360° scan of a subject. FIG. 5 is an illustration of a scan acquisition sequence wherein the subject is positioned on a rotating surface and rotated while the camera is stationary. In one embodiment, the camera can be transported in a circle around a stationary subject, e.g. with a motor or lever.

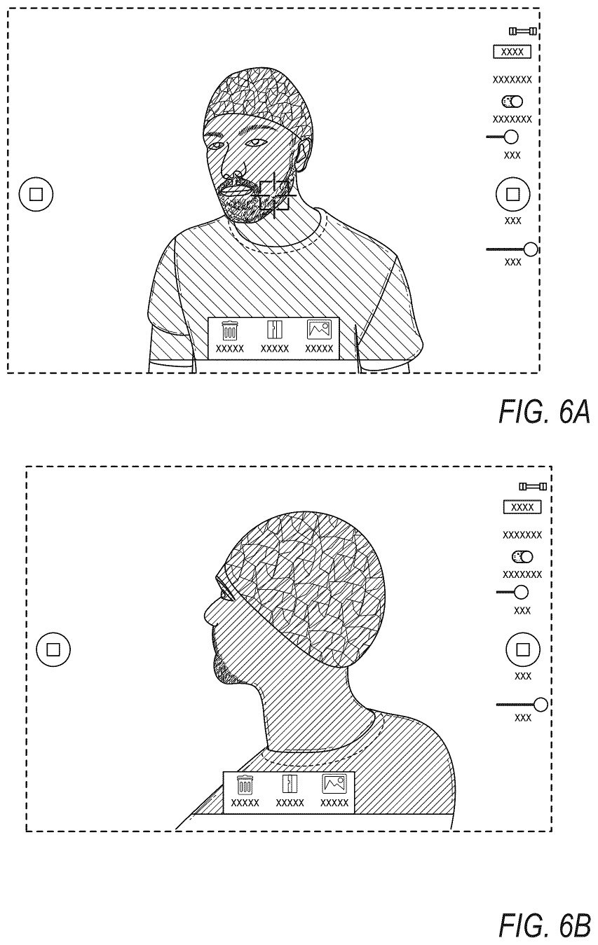

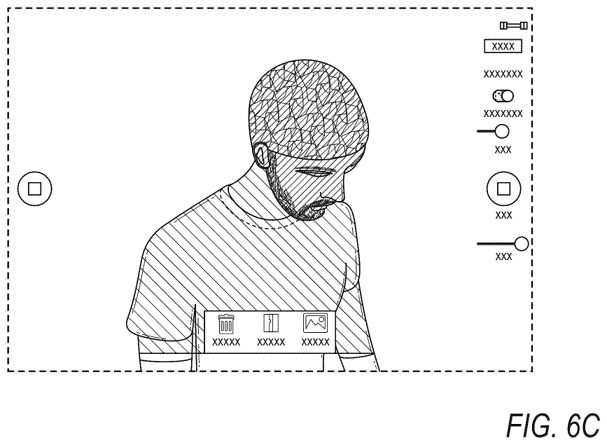

In one embodiment, the scan acquisition process can include displaying data in a user interface. The user interface can be a data collection interface that includes feedback regarding the scan acquisition. FIG. 6A, FIG. 6B, and FIG. 6C are illustrations of a user interface according to one example. The data displayed in the user interface can include an indicator of a center or focal point of the camera field of view. In one embodiment, the data displayed can include guidance for a position of the camera or the subject. In one embodiment, the user device can display the depth values acquired in the scan. For example, FIGS. 6A-6C illustrate depth values as a depth map of gradient colors. In one embodiment, the user device can display a warning when the user device is outside of a range of distance relative to the subject. For example, the user device can be too close to the subject or too far from the subject (as in FIG. 6C). When the user device is outside of the range of distance, it may be difficult to acquire accurate or sufficient scan data. In one embodiment, the user device can receive depth data from the depth sensors and can process the depth data in real time in order to display the user interface.

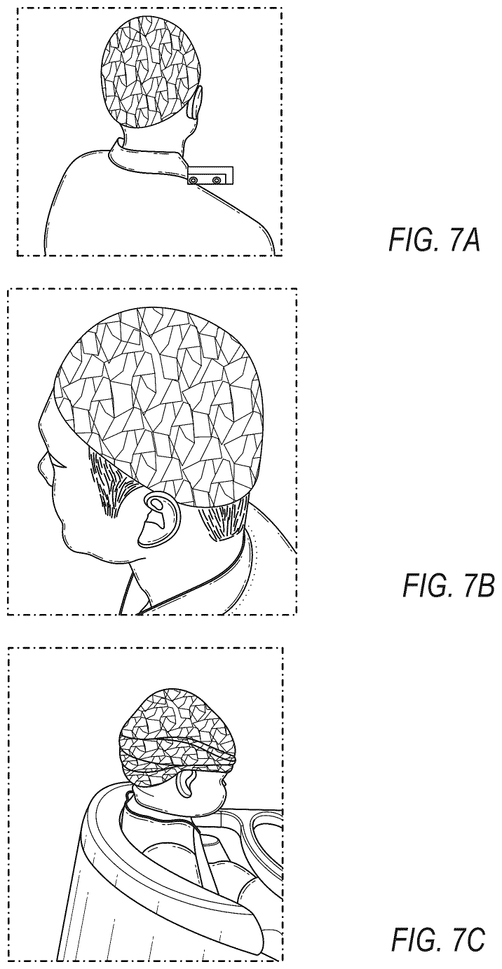

In one embodiment, the scan acquisition can include acquiring scan data of a subject wearing a cap. The cap can be useful for hiding hair artifacts. In one embodiment, the cap can include visual features such as a pattern or shapes. FIG. 7A, FIG. 7B, and FIG. 7C are illustrations of caps that can be worn by the subject. As an example, the cap can be a thin, opaque textile material. The visual features of the cap can provide landmarks or feature points that can be identified during image processing in order to improve the accuracy of image processing. Advantageously, the visual features of the cap do not need to be positioned at specific pre-set locations on the subject. For example, the cap does not to be precisely placed in a certain position or orientation on the subject. The presence of the visual features on the cap, without known or pre-set locations of the visual features, can be sufficient for image processing.

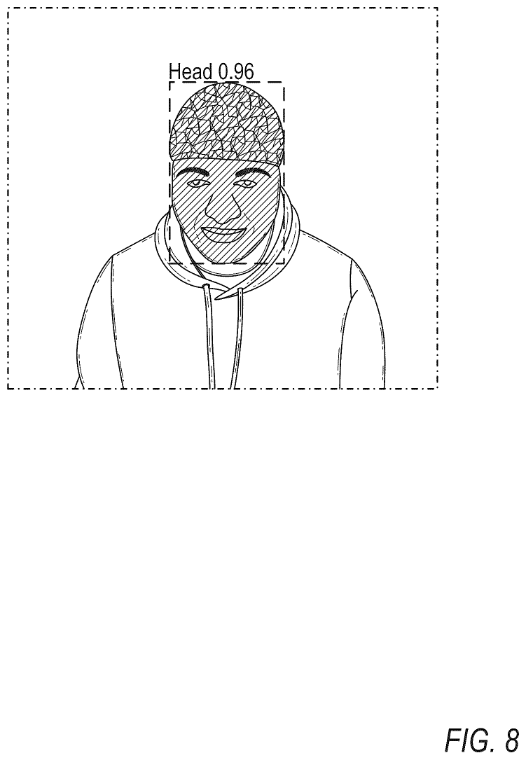

In one embodiment, foreground estimation can include identifying and isolating pixels that make up a region (or plane) of interest as the foreground of an image. The head of a subject is used herein as a non-limiting example of a region of interest. In one embodiment, the foreground estimation can be performed using a machine learning or artificial intelligence model. For example, an object detection neural network can be used to identify the pixels that make up a region of interest. In one embodiment, the machine learning model can be trained to identify a specific body part as the region of interest. FIG. 8 is an illustration of foreground estimation wherein the head is the region of interest. The pixels that make up the head can be identified and segmented from the remainder of the image in a bounding box. The identified pixels can be covered by a segmentation mask. In one embodiment, the foreground estimation can include determining a confidence or certainty level associated with the identified pixels. In one embodiment, the mask can be processed to improve continuity of the mask. For example, gaps between pixels or disconnected pixels can be removed by adding or removing pixels from the mask.

In one embodiment, the foreground estimation can include receiving an input related to identifying the region of interest. For example, a user device can receive an input that identifies pixels in the region of interest. In one embodiment, a user device can receive an input to correct or update an identified region of interest. For example, the user device can use a machine learning model to identify an initial region of interest. The user device can then receive an input to modify the initial region of interest by adding or removing pixels. In this manner, the foreground estimation can include incorporation of manual input.

Foreground estimation can improve the accuracy of subsequent image processing steps. For example, feature extraction steps can be limited to the region of interest that is identified by foreground estimation for greater accuracy. The background of the image, which can be especially noisy when an image is captured in a non-clinical environment, can be ignored after foreground estimation.

In one embodiment, the region of interest that is identified in foreground estimation can be analyzed and/or processed to ensure that the image can be used for 3D reconstruction. The analysis can include any combination of camera displacement analysis, blur analysis, depth map analysis, feature matching, loop closure analysis, surface coverage analysis, and landmark analysis. In one embodiment, any of the analysis steps can include processing based on the analysis to improve the image quality. In one embodiment, any of the analysis steps can be performed during a scan to provide data that can be used to improve the scan quality.

In one embodiment, the camera displacement analysis can include estimating a displacement (or motion) of the camera between frames of the scan. In one embodiment, the displacement of the camera can be determined photogrammetrically. In one embodiment, the displacement of the camera can be determined based on two-dimensional image data. The image data can be color (red-green-blue, RGB) data. In one embodiment, the displacement analysis can be performed using depth information. The photogrammetric determination of camera displacement can include identifying common points or features between two sequential images and determining a relative displacement of the common points that is caused by displacement of the camera. These features can be in two-dimensional image space, or in three-dimensional space as a point cloud. Camera displacement can be estimated between one or more pairs of frames in the scan.

In one embodiment, an estimated camera displacement can be compared with a displacement threshold to determine whether the camera displacement is acceptable. When the camera displacement is too large, it is possible that the sequential frames may not share enough features for 3D reconstruction. In one embodiment, when the estimated camera displacement is greater than a displacement threshold, the user device can generate a notification (e.g. a display) to repeat at least a portion of the scan with smaller camera displacement between frames.

In one embodiment, the blur analysis can include calculating a blur metric of an image. The blur metric can quantify non-uniform blur, such as motion blur, or uniform blur. In one embodiment, the blur metric can be calculated based on a single image without use of a reference image because the scan may not include images that are suitable as reference images. In one embodiment, the blur metric can be calculated as described in Javaran, Taiebeh Askari, Hamid Hassanpour, and Vahid Abolghasemi. “A noise-immune no-reference metric for estimating blurriness value of an image.” Signal Processing: Image Communication 47 (2016): 218-228, which is incorporated herein by reference in its entirety. In one embodiment, the motion blur can be quantified as a matrix representing optical flow between consecutive frames. The motion may be removed by convolving the image with an inverse of a motion matrix.

In one embodiment, the blur analysis can include correcting blur in an image. For example, the blur metric can be compared to a blur metric threshold in order to determine whether the quality of the image is acceptable, and a motion correction can be applied when the blur metric is greater than the blur metric threshold. In one embodiment, the motion correction can include inputting the image to a neural network, e.g. a convolutional neural network (CNN). The neural network can be trained to remove blur from the image and output a deblurred image.

In one embodiment, the blur metric calculation and motion correction can be repeated to improve image quality. In one embodiment, if the blur metric is still too high after one or more motion correction applications or if the change in blur metric is small after motion correction, the image can be excluded from further analysis.

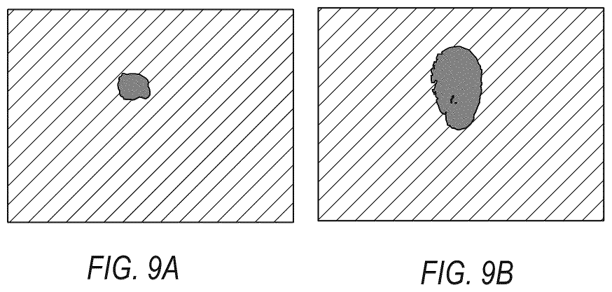

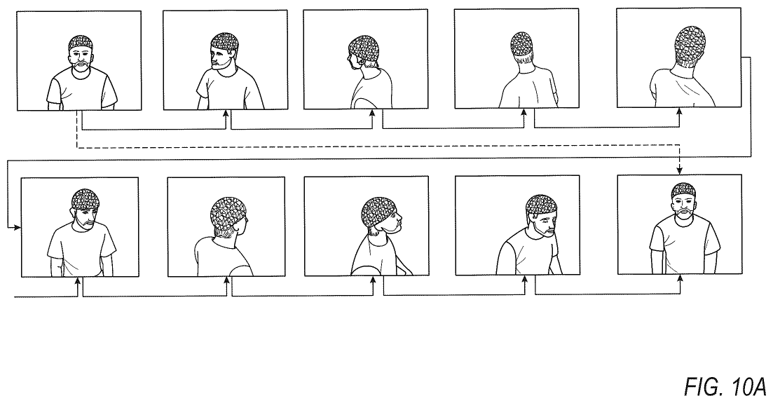

In one embodiment, the depth map analysis can include determining whether a region of interest includes a certain number of pixels having corresponding non-zero depth values. If the region of interest does not have enough non-zero depth values, it may not be suitable for 3D reconstruction because the depth of the pixels cannot be determined. FIG. 9A illustrates an example of an incomplete depth map that does not have enough non-zero pixels, and FIG. 9B illustrates an example of a complete depth map that has enough non-zero pixels. In one embodiment, the number of pixels with non-zero depth values can be compared to a threshold. If the number of pixels is less than the threshold, the image can be excluded from further analysis.

Depth data can be compromised due to movement of the camera or subject. It is therefore possible that a number of sequential (or consecutive) frames have insufficient depth data, and removing all of the sequential frames would result in a gap in the 3D reconstruction. Accordingly, in one embodiment, depth maps of a group of images can be analyzed in order to determine whether the group of images should be excluded from further analysis. In one embodiment, an average number of pixels having non-zero depth values for the group of images can be calculated. Images in the group that have fewer than k standard deviations less than the average can be excluded from further analysis. The variable k can vary and can be any number. For example, k=1, k=1.5, k=2, etc. In this manner, images can be removed based on their deviation from surrounding images rather than an absolute threshold. Preserving surrounding images based on the average number of pixels in the group can prevent creating a large gap in the image data. In one embodiment, the user device can generate a notification (e.g. a display) when a number of images are removed, e.g. when the number of images that are removed is greater than a second threshold.

In one embodiment, the feature matching can include identifying features in two-dimensional image data and determining whether there are matching features across consecutive images. The presence of matching features or a certain number of matching features can be used to register (align) consecutive images with each other during 3D reconstruction.

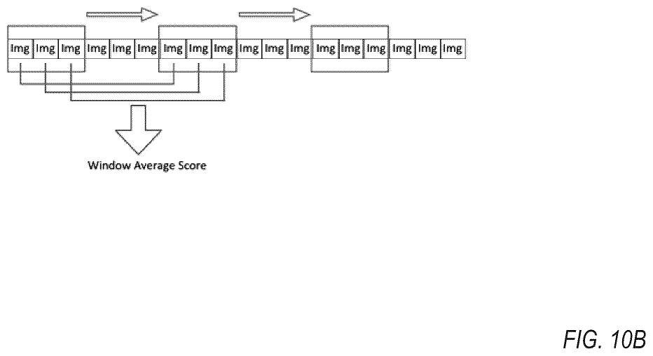

In order to be used for 3D reconstruction, the scan should include a full (closed) loop around the subject. In one embodiment, the loop closure analysis can include identifying matching features that are present in a number of initial images in the scan and in a number of final images in the scan. Matching features between the initial images and the terminal images in the scan can indicate that the camera has closed the loop during the scan and is in the same position in the beginning of the scan and at the end of the scan. In one embodiment, they can also be used to optimize and correct camera pose estimations throughout the scan. In one embodiment, the scan can be determined to include a full loop when a certain number of matching features are present between the initial images and the final images and/or when the locations of matching features are aligned in the initial images and the final images. FIG. 10A is an illustration of a sequence of images representing loop closure, wherein the first and the last image are approximately aligned and include matching features that can be used to calculate camera pose displacement between these frames directly to compare with the camera displacement that has been consecutively calculated throughout previous frames. The subject and the body part of interest, the head, are registered (aligned) across all the images in the loop. In one embodiment, groups of images can be compared to identify matching features, as illustrated in FIG. 10A. In one embodiment, a filter can be used to identify the matching features.

In one embodiment, the identification of matching features for loop closure analysis can be performed while the scan is being acquired. In one embodiment, when matching features from initial images are identified in later images, the scan can be automatically terminated because the loop has been closed. In one embodiment, the initial images and terminal images containing matching features may not be the initial and terminal images of the scan. The images comprising the closed loop may be a subset of images in the scan. In one embodiment, the subset of images can be identified based on matching features. In one embodiment, a method for determining loop closure can include calculating an average comparison score for two subsets (windows) of sequential images, e.g. each window including a number of sequential images. Two windows can be compared by comparing the first image in the first window with the first image in the second window; the second image in the first window with the second image in the second window; the third image in the first window with the third image in the second window; etc. The images having the same index or position in the respective windows can be compared to identify matching features. Each comparison can result in a score to quantify the number of matching features. The scores can be averaged across a window to generate a window average score. A high window average score, e.g., a window average score above a threshold, can indicate that the loop is closed because there are matching features across sequential images. FIG. 10B is an illustration of windows of images that can be compared with each other to generate a window average score. In one embodiment, the window average score can be repeatedly calculated across different windows until loop closure is detected. Each new window used for comparison can be overlapping or non-overlapping with a previous window used for comparison.

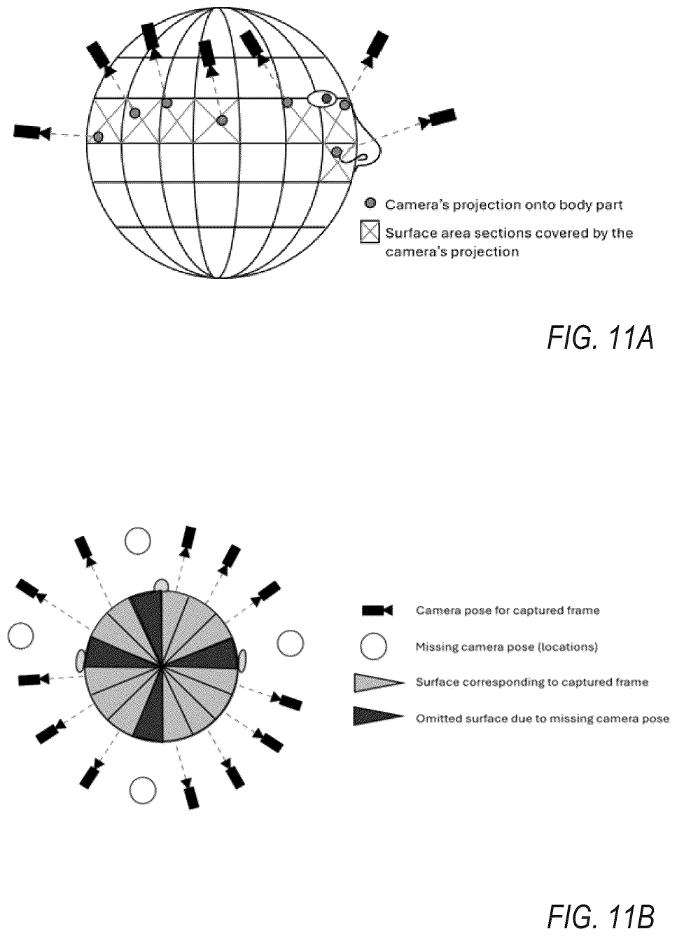

In one embodiment, the surface coverage analysis can include estimating poses of the camera during the scan and determining surface areas that are captured by the scan based on the estimated poses. Each frame acquired by the camera can be mapped to a limited surface area based on the position and orientation of the camera in three-dimensional space. FIG. 11A is an illustration of surface areas corresponding to different camera poses on a representation of a body part (a sphere). The spherical representation, which can represent a head, can be divided into sections or tiles with latitude and longitude lines. In one embodiment, the camera pose analysis can be displayed using an avatar of the body part by visually indicating surface area coverage on the avatar. Each camera pose can correspond to a surface area covering one or more tiles, or portions of a tile. In this manner, the surface coverage corresponding to the scan data can be quantified (e.g., as a number of tiles, a percentage of available tile surface area that is covered, a trajectory or shape of covered tiles) and assessed. For example, the trajectory of the camera, as represented by the covered surface area, can be compared to a target trajectory to ensure whether the scan has acquired sufficient coverage of the entire body part.

In one embodiment, the camera pose relative to the subject's body part can be estimated using neural network-based coordinate regression. A neural network can be trained to predict 3D coordinates corresponding to pixels of a given image. In one embodiment, the prediction can include angle regression based on the roll, pitch, or yaw angles of the camera during the scan to accurately determine the pose of the camera in 3D space.

FIG. 11B is a top-view illustration of a 3D representation of a head with surface areas that are covered and surface areas that are not covered by a scan. Missing camera poses result in omitted surface areas which may be needed for a full 3D representation. A camera pose may be missing because the scan was too fast or because there was too much blur in an image. Determining the surface area coverage can be useful for determining whether more scan data is needed.

In one embodiment, the landmark analysis can be an initial landmark analysis based on 2D image data. The initial landmark detection can include determining whether landmarks are visible in a number of scan images. As an example, landmarks in a scan of a head can include facial features such as the nose, eyes, and ears, or cephalometric landmarks such as the tragion, nasion, basion, gnathion, etc. In one embodiment, the initial landmark detection can be performed using a machine learning model. In one embodiment, when landmarks are not visible in the 2D image data, the user device can generate a notification (e.g. a display) to repeat at least a portion of the scan.

In one embodiment, the scan data can be downsampled prior to 3D reconstruction. The downsampling can include selecting images that meet certain quality metrics, for example, based on the analysis steps described above, and excluding other images from further analysis. In one embodiment, the quality metrics can include a number of landmarks detected or a camera pose to ensure that necessary angles of the head are covered by the selected frames. In one embodiment, the selection criteria of an image can be based in part on the index of the image in the scan. For example, an early image can be evaluated for downsampling in a different manner than a later image to ensure that features in an image are retained from all angles (positions) around the head.

In one embodiment, the estimation of camera displacement can be used to adaptively collect and/or downsample images at a higher rate when the displacement is small. When the camera displacement is small between frames, the relative head motion is small or slow, and fewer images are needed to be retained for 3D reconstruction. When the camera displacement is larger, more or all images can be retained to compensate for the larger relative head motion between frames and increase the likelihood that consecutive frames include matching features.

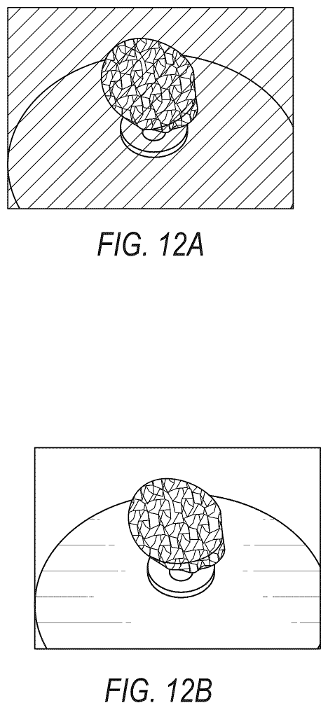

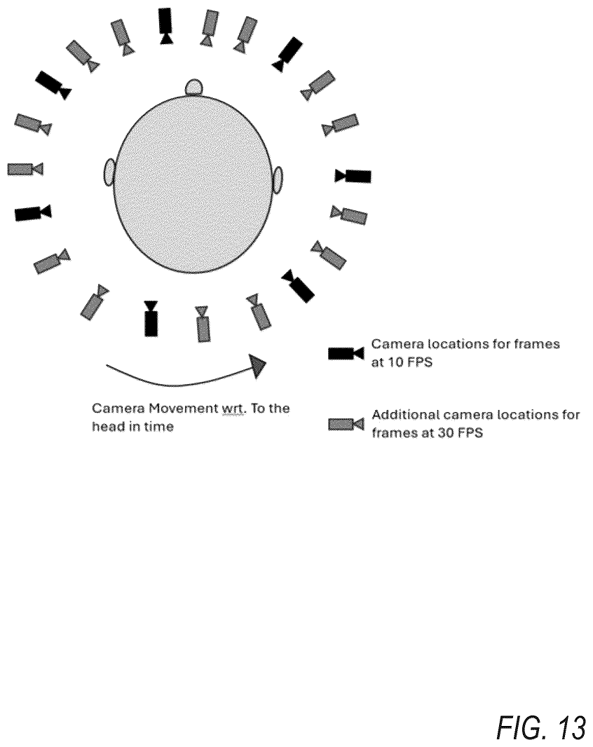

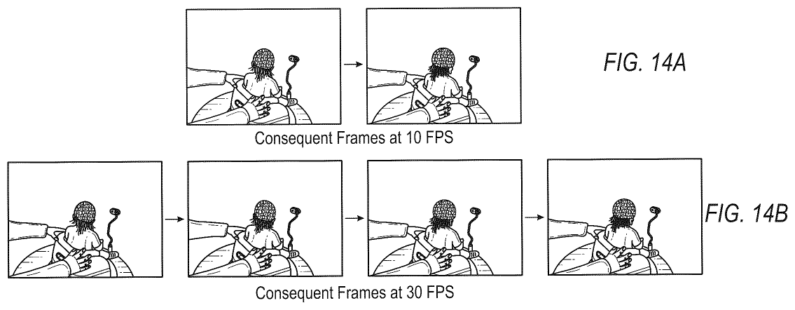

In one embodiment, motion compensation can be applied to the scan data. Motion compensation can include reducing motion blur in frames and applying an adaptive frame rate to reduce the probability of omitting features of interest or certain surface areas of the body part. In one embodiment, motion compensation can include adjusting scan data acquisition parameters such as exposure time and frame rate. A short exposure time can mitigate blur due to motion, and can be accompanied by high light sensitivity (ISO) to preserve brightness. FIG. 12A is an example of an image acquired with longer exposure time having more blur than FIG. 12B, which is an example of an image acquired with shorter exposure time. In one embodiment, a high frame rate (frames per second or FPS) can reduce omissions in the scan data. FIG. 13 is an illustration of how an increased frame rate can result in scan data acquisition from more camera poses, resulting in increased surface area coverage. FIG. 14A illustrates a number of frames (2) that are acquired with a frame rate of 10 FPS, while FIG. 14B illustrates a number of frames (4) that are acquired with a frame rate of 30 FPS.

In one embodiment, motion compensation can include using a neural network or other machine learning model to generate a deblurred image, e.g. as described above. In one embodiment, motion compensation can be applied in conjunction with calculation of a blur metric in order to quantify the efficacy of deblurring and improve the efficiency of the system by only applying motion compensation to images with high blur metrics.

In one embodiment, an adaptive frame rate can be applied by downsampling images after a scan is acquired. In one embodiment, the downsampling rate can be based on motion blur in an image (e.g. a blur metric). When there is a high level of motion blur in one or more sequential images, the downsampling can be reduced in order to avoid omitting necessary image data. In one embodiment, the downsampling rate can be based on camera pose estimation. The camera pose for each frame that is selected for processing can be used to determine whether the frames comprise a target camera trajectory for 3D reconstruction. If the camera pose changes quickly, the downsampling rate can be reduced in order to avoid omitting necessary image data.

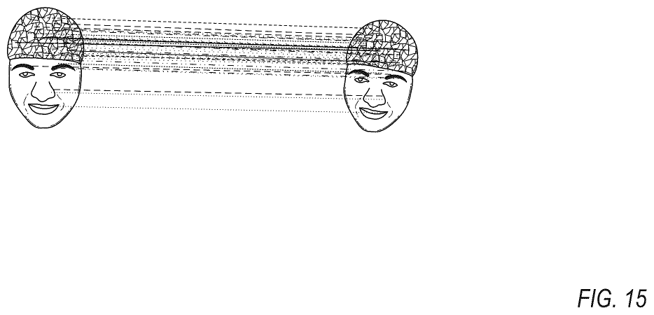

The mesh generation step 2400 can include one or more image processing steps, including camera pose estimation based on points of interest, camera pose optimization, and point cloud registration. Camera pose estimation can be performed based on points of interest (or features of interest) that are expected to retain their appearance in different frames. These points can be, for example, visual features on a cap that the subject is wearing or facial features. In one embodiment, the points of interest can be identified in images using methods for feature detection such as Scale Invariant Feature Transform (SIFT), Oriented FAST and Rotated BRIEF (ORB), machine learning methods, trained neural networks, etc. In one embodiment, matching points of interest across images (e.g. sequential images) can be matched, as illustrated in FIG. 15. In one embodiment, the depth map of each image can be used to determine the 3D positions of the points of interest with respect to the camera coordinate system. Since the points of interest are located on a body part (e.g. the head), it can be assumed that the points do not move relative to the body part. Accordingly, any transformation in 3D space of the points between images can be attributed to transformation of the camera relative to the head. The transformations of the points between images can be used to track the camera's pose between images. In one embodiment, the camera's pose can be determined relative to an initial pose, which can be, for example, the pose associated with an initial image of the scan.

In one embodiment, error correction can be applied to the determined camera poses. Small errors in each calculated camera pose can propagate to further camera pose calculations since the pose calculations are dependent on each other in a kinematic chain. In one embodiment, the detection of loop closure between one or more initial images and one or more terminal images of a scan can be used to calculate and correct error in the determined camera poses. For example, a transformation in camera pose between the initial images and the terminal images that represent a closed loop can be directly calculated because the initial images and the terminal images have matching features and feature positions. The direct transformation between the initial images and the terminal images can be compared with a transformation as calculated throughout the entire loop. In one embodiment, the direct transformation can be used in place of the transformation throughout the loop. In one embodiment, a weighted average of the two transformations can be used.

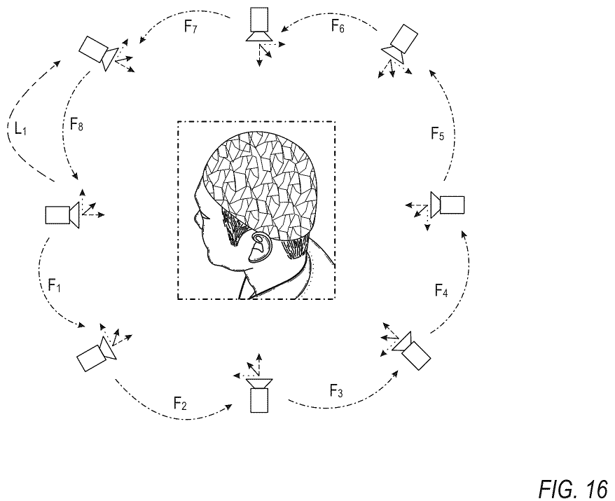

FIG. 16 is an illustration of calculated camera poses across a number of frames in a loop. The position of the camera at the terminal frame (F8), as calculated based on transformation through the entire loop from F1 through F7, can be corrected by calculating a direct transformation L1 between the initial frame (F1) and F8.

In one embodiment, the terminal image(s) indicating loop closure can be identified using a machine learning model (e.g. a neural network). For example, a scan can begin and terminate with a frontal (direct) view of the subject's face. A facial recognition model can be used to detect when a face is present in an image. The facial recognition can include, for example, the orientation of the face. The facial recognition model can be applied to images in a scan to determine a final image wherein a face is present and in direct view in a similar manner to an initial image of the scan. In one embodiment, global optimization or bundle adjustment can be applied to determine when the loop has been closed based on alignment of images, as described in Choi, Sungjoon & Zhou, Qian-Yi & Koltun, Vladlen. (2015). Robust Reconstruction of Indoor Scenes. 10.1109/CVPR.2015.7299195, which is incorporated herein by reference in its entirety for all purposes.

In one embodiment, the pixels in a region of interest (in the foreground) can be projected into 3D space based on corresponding depth values in a depth map to generate a point cloud. In one embodiment, a relationship between the coordinates of the pixels in 3D space in the point cloud and a real-world coordinate system can be determined based on the estimated camera poses. For example, the relationship can be quantified by a transformation or mapping. Point clouds that are captured from different camera poses can be merged based on their corresponding real world coordinates. In one embodiment, the point cloud can be downsampled. In one embodiment, points in the point cloud that do not have a sufficient (e.g. threshold) number of neighboring points within a given radius can be removed from the point cloud, which can remove erroneous artifacts, or to avoid gaps.

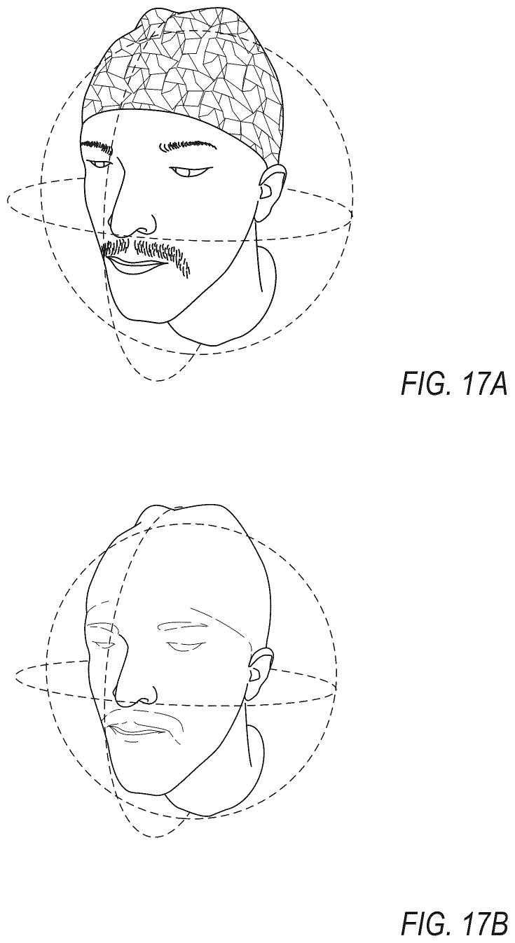

In one embodiment, a mesh generation algorithm can be applied to the merged point cloud to generate a mesh surface that represents the body part. Mesh generation algorithms can include, but are not limited to, Poisson surface reconstruction, Alpha shape reconstruction, or ball pivoting algorithms. FIG. 17A is an illustration of a mesh surface with texture generated according to the present disclosure, and FIG. 17B is an illustration of a mesh surface without texture generated according to the present disclosure.

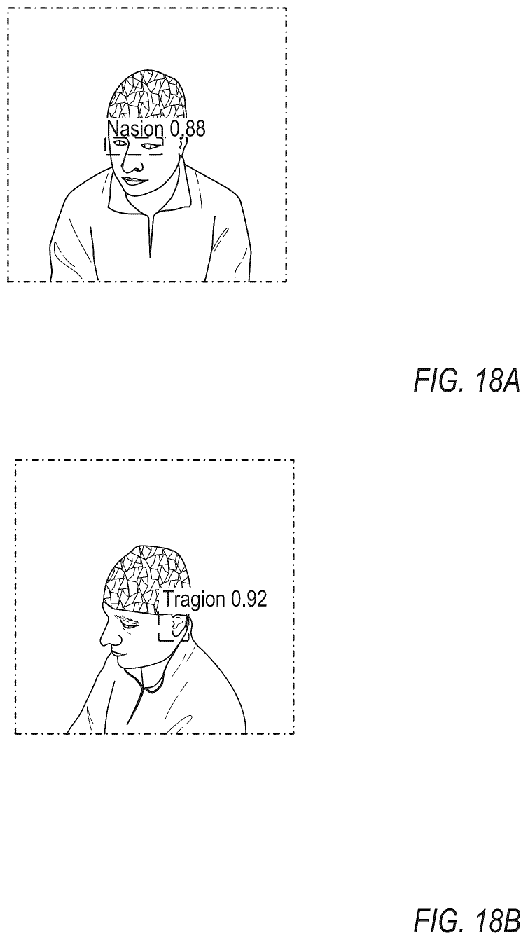

In one embodiment, landmarks (e.g. the nasion, left and right tragion) can be detected in 2D or 3D space based on the RGB images and depth maps. In one embodiment, the pixels that correspond to landmarks can be determined by a machine learning model (e.g. a deep learning model). FIG. 18A is an illustration of a detected nasion on an RGB image, and FIG. 18B is an illustration of a detected tragion on an RGB image. In one embodiment, the machine learning model can generate bounding boxes around the detected landmarks. In one embodiment, the machine learning model can have a YOLO architecture. The model can be applied to the isolated region of interest for greater accuracy.

In one embodiment, the landmarks can be detected with directionality. For example, a detected tragion can be distinguished as a left side or right side tragion. In one embodiment, the tragion side can be determined based on the relative positions of the tragion and a detected nasion, or, more generally, a detected nose and ear including the nasion and tragion, respectively. For example, when the head is upright in an image and the ear bounding box is to the left of the nose bounding box in the image, the tragion is on the right side of the subject, and when the ear bounding box is to the right of the nose bounding box in the image, the tragion is on the left side of the subject.

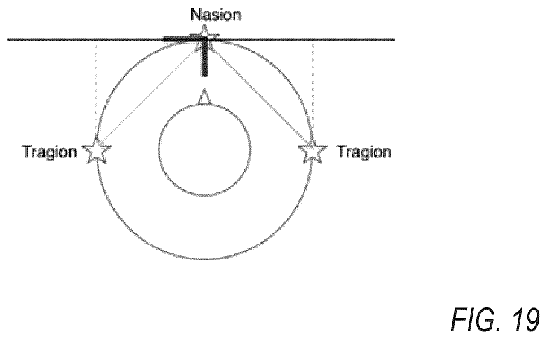

In one embodiment, when a nose bounding box is not present in the same frame as the ear/tragion, a different method can be applied to determine the tragion side. FIG. 19 is an illustration of the relative positions or directions of tragions and nasions. In one embodiment, the method can include identifying two tragion clusters, one being a right tragion and one being a left tragion. In order to determine which tragion is right or left, the direction of nasion movement as the scan progresses can be detected. As the scan progresses, the nose moves into and across the field of view of the camera. Determining the direction from which the nose enters the field of view, as well as the location of the nasion relative to the tragions, can be used to determine the direction of the scan and therefore the sides of the tragions. For example, when the nasion moves clockwise in consecutive images and the nasion is between the two detected tragion regions, the first (leftmost) tragion in the image can be the right tragion of the subject's head and the second (rightmost) tragion in the image can be the left tragion of the subject's head.

In one embodiment, the tragion side can be determined based on the camera pose during acquisition of images including a tragion. The camera pose associated with the tragions can be compared to a reference camera pose during acquisition of images including the nasion. The difference in the poses can be used to determine the tragion sides. For example, when the camera is to the left of the reference pose during acquisition of a tragion, the tragion is likely the right tragion.

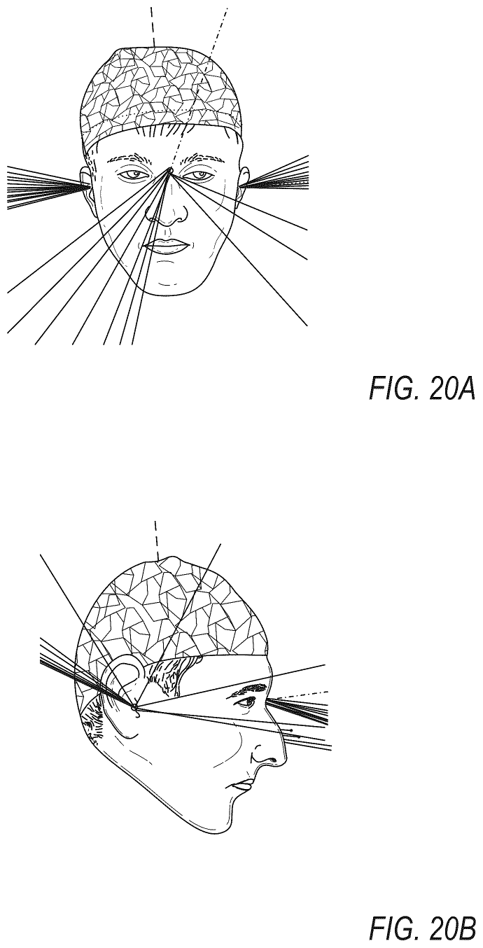

In one embodiment, an initial projection of the pixels of the detected landmarks into 3D space can be made based on the corresponding depth map values. In one embodiment, a direction vector can be calculated between the projected position and a camera pose in 3D space as determined by camera pose estimation techniques described herein. The direction vector can then be applied from the camera pose to the generated mesh, in one embodiment using hit testing, in order to determine a point on the mesh surface that corresponds to the projected position and the camera pose. Hit testing can refer to identifying a space on the mesh surface and determining whether a point (e.g. the vector endpoint) falls within the space. FIG. 20A and FIG. 20B are illustrations of points on a mesh surface that correspond to landmarks identified in the 2D images used to generate the mesh surface. In one embodiment, for an identified landmark, one point representing the landmark can be generated per frame and then projected onto the mesh surface to generate a 3D point cloud of the landmarks. In one embodiment, a centroid or median coordinate of the landmark point cloud can be the representative location of the landmark. In one embodiment, landmarks can be directly identified from the generated mesh volume using a machine learning model. For example, a neural network can be used to detect landmarks on a mesh surface.

In one embodiment, cranial parameters can be determined based on the identified landmarks on the mesh surface. Cranial parameters can include, for example, cephalic index (CI), cranial vault asymmetry (CVA), and cranial vault asymmetry index (CVAI). CI refers to a ratio of the head width to the head length while CVAI refers to a ratio of asymmetry between a right diagonal and a left diagonal, or a first diagonal and second diagonal, and can be defined as the ratio of CVA divided by the larger of the first diagonal and the second diagonal, where CVA is defined by subtracting the maximum and minimum diagonals of the head in top view. Comparisons of these cranial parameters to benchmark values can indicate a condition of a patient. For example, an infant's head can be diagnosed with mild plagiocephaly when CVAI is greater than 3.5%. An infant's head can be diagnosed with brachycephaly when CI is larger than 90%. An infant's head can be diagnosed with scaphocephaly when CI is less than 76%.

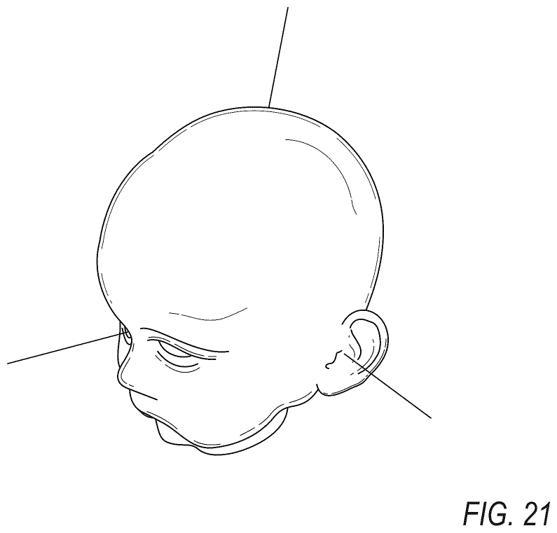

The cranial parameters can be determined based on the identified nasion and tragion landmarks in the 3D mesh surface. The landmarks can define a coordinate system for calculating the cranial parameters. For example, FIG. 21 illustrates a coordinate system defined by the tragions and nasion. A vector from the left tragion to the right tragion can be normalized to define a y-axis of a coordinate system, wherein the midpoint of the vector is the origin of a coordinate system. The vector from the origin to the nasion can be normalized to define the x-axis. The cross product of the x- and y-axes can define the z-axis.

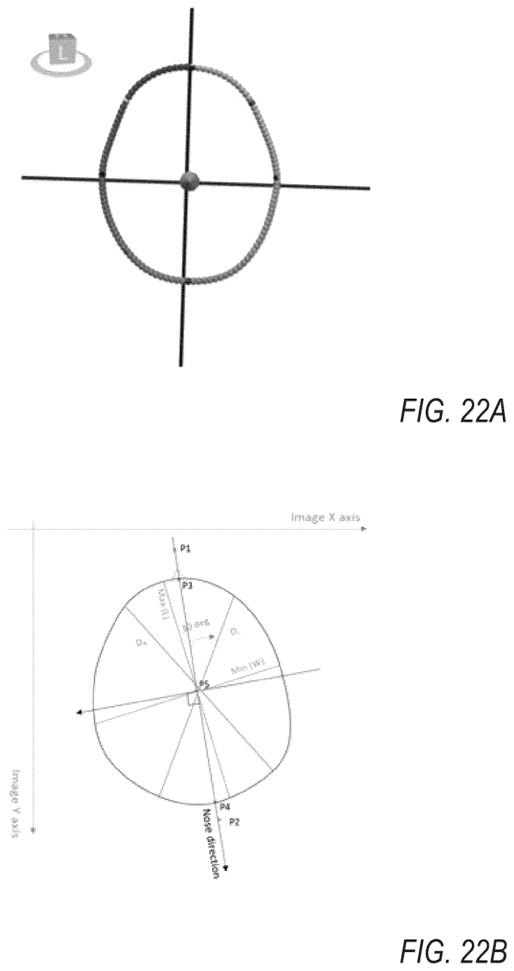

In one embodiment, cranial parameters can be determined by approximating a cross-section of the head at a given height (position along the z-axis). As an example, the given height can be a height corresponding to a maximum head circumference above the ears. The maximum head circumference above the ears can be reported as the head circumference. In one embodiment, the determination of the head circumference and/or cranial parameters can include performing a hit test at angular intervals (e.g. 1° intervals, 3° intervals) in the x-y plane (orthogonal to the z-axis). The hit tests can be performed to determine points of the mesh surface at each angular interval. The points then form a contour of the head in the x-y plane at the given height. FIG. 22A is an illustration of a head contour represented as a polygon of 360 points in the x-y measurement plane. FIG. 22B is an illustration of the head contour and key points or segments used to calculate cranial parameters. For example, head length can be measured as the distance between 0° and 180° along the contour, the head width can be determined as the distance between 90° and 270° along the contour, the right diagonal can be determined as the distance between 40° and 220° along the contour, and the left diagonal can be determined as the distance between 140° and 320° along the contour. The segments can then be used to calculate cranial parameters. In one embodiment, the contour can be smoothed using a smoothing algorithm, e.g. a Savitsky-Golay filter.

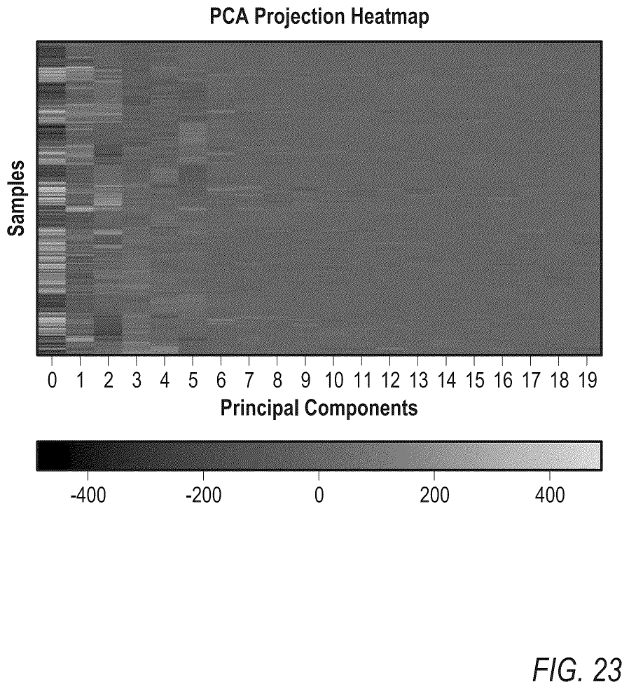

In one embodiment, the cranial parameters and the mesh surface of the head can be analyzed to diagnose conditions such as craniosynostosis and deformational plagiocephaly/brachycephaly (DPB). In one embodiment, principal component analysis (PCA) or a signed distance function (SDF) can be applied to process the 3D mesh surface as a vector. The vector can then be analyzed using regression analysis (e.g. comparing with other vectors associated with normal or deformed heads) or a machine learning model that is trained on other vectors associated with normal or deformed heads to determine whether the head is deformed. FIG. 23 is a heatmap of different cranial shapes represented by principal components, where each row represents a certain cranial shape.

Table 1 illustrates classification accuracy for determinations of manifestations of craniosynostosis and synostotic vs. non-synostotic deformities for 94 synostotic and 396 non-synostotic patients according to one embodiment of the present disclosure.

TABLE 1

| Accuracy for: | |
|---|---|
| Left Unicoronal | 90% |
| Right Unicoronal | 89% |
| Sagittal | 93% |
| Metopic | 91% |
| Average Inter-Class Prediction | 89% |
| Accuracy | |
| Synostotic vs Not Prediction | 98% |

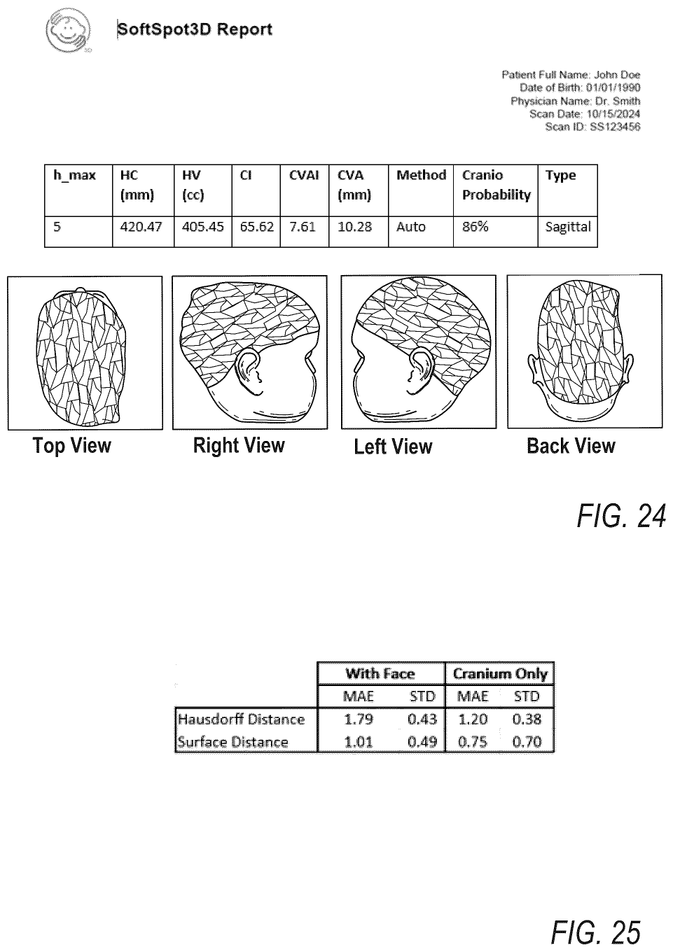

FIG. 24 illustrates a sample report that can be generated based on the 3D reconstruction methods and analysis methods described herein according to one embodiment of the present disclosure. As an example, the surface mesh of the head and the cranial parameters can be determined. FIG. 25 illustrates the surface distance accuracy (mean absolute error (MAE) and standard deviation (STD)) of the 3D reconstruction for the whole head (with face) and for an isolated cranium according to one embodiment of the present disclosure for 45 patients aged 3-12 months.

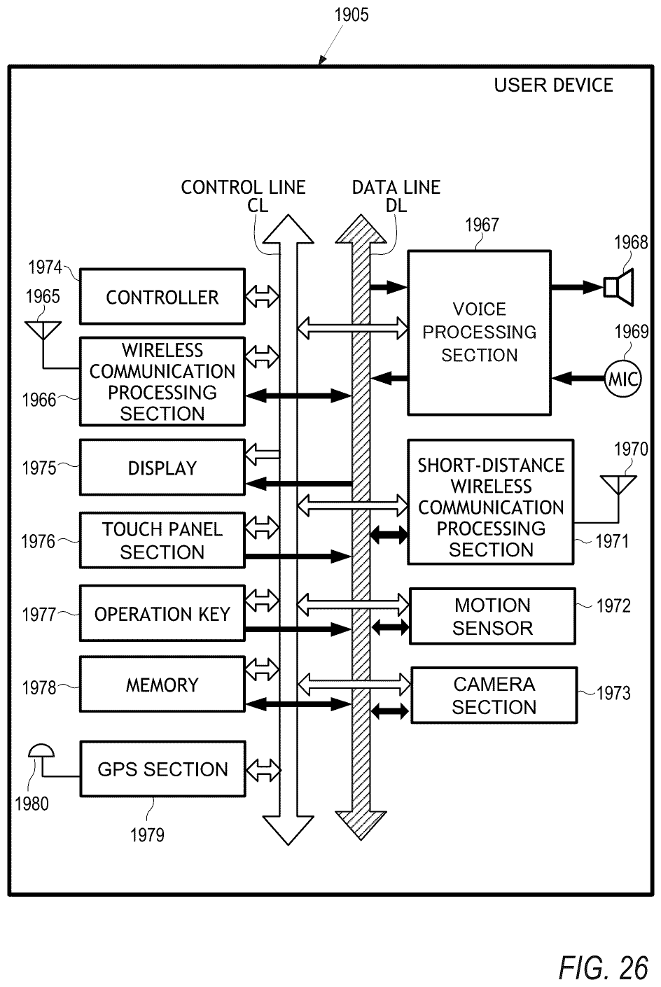

FIG. 26 is a more detailed block diagram illustrating a user device 1905, or mobile device, according to one embodiment of the present disclosure. In one embodiment, user device 1905 may be a smartphone. However, the skilled artisan will appreciate that the features described herein may be adapted to be implemented on other devices (e.g., a laptop, a tablet, a server, an e-reader, a camera, a navigation device, etc.). The user device 1905 of FIG. 26 includes a controller 1974 and a wireless communication processor 1966 connected to an antenna 1965. A speaker 1968, or an output device, and a microphone 1969 are connected to a voice processor 1967.

The controller 1974 is an example of a control unit and may include one or more central processing units (CPUs) and/or one or more graphics processing units (GPUs), and may control each element in the user device 1905 to perform functions related to communication control, audio signal processing, control for the audio signal processing, still and moving image processing and control, and other kinds of signal processing. The controller 1974 may perform these functions by executing instructions stored in a memory 1978. Alternatively or in addition to the local storage of the memory 1978, the functions may be executed using instructions stored on an external device accessed on a network or on a non-transitory computer readable medium.

The memory 1978 is an example of a storage unit and includes but is not limited to Read Only Memory (ROM), Random Access Memory (RAM), or a memory array including a combination of volatile and non-volatile memory units. The memory 1978 may be utilized as working memory by the controller 1974 while executing the processes and algorithms of the present disclosure. Additionally, the memory 1978 may be used for long-term storage, e.g., of image data and information related thereto. As disclosed above, the memory 1978 may be configured to store longitudinal patient information including anatomical measurements or, in an example, cranial parameters.

The user device 1905 includes a control line CL and data line DL as internal communication bus lines. Control data to/from the controller 1974 may be transmitted through the control line CL. The data line DL may be used for transmission of voice data, display data, etc.

The antenna 1965 transmits/receives electromagnetic wave signals between base stations for performing radio-based communication, such as the various forms of cellular telephone communication. The wireless communication processor 1966 controls the communication performed between the user device 1905 and other external devices via the antenna 1965. For example, the wireless communication processor 1966 may control communication between base stations for cellular phone communication.

The speaker 1968 emits an audio signal corresponding to audio data supplied from the voice processor 1967. The microphone 1969 detects surrounding audio and converts the detected audio into an audio signal. The audio signal may then be output to the voice processor 1967 for further processing. The voice processor 1967 demodulates and/or decodes the audio data read from the memory 1978 or audio data received by the wireless communication processor 1966 and/or a short-distance wireless communication processor 1971. Additionally, the voice processor 1967 may decode audio signals obtained by the microphone 1969.

The user device 1905 may also include a display 1975, a touch panel 1976, an operation key 1977, and a short-distance wireless communication processor 1971 connected to an antenna 1970. The display 1975 may be a Liquid Crystal Display (LCD), an organic electroluminescence display panel, or another display screen technology. In addition to displaying still and moving image data, the display 1975 may display operational inputs, such as numbers or icons which may be used for control of the user device 1905. The display 1975 may additionally display a GUI for a user to control aspects of the user device 1905 and/or other devices. Further, the display 1975 may display characters and images received by the user device 1905 and/or stored in the memory 1978 or accessed from an external device on a network. For example, the user device 1905 may access a network such as the Internet and display text and/or images transmitted from a Web server.

The touch panel 1976 may include a physical touch panel display screen and a touch panel driver. The touch panel 1976 may include one or more touch sensors for detecting an input operation on an operation surface of the touch panel display screen. The touch panel 1976 also detects a touch shape and a touch area. Used herein, the phrase “touch operation” refers to an input operation performed by touching an operation surface of the touch panel display with an instruction object, such as a finger, thumb, or stylus-type instrument. In the case where a stylus or the like is used in a touch operation, the stylus may include a conductive material at least at the tip of the stylus such that the sensors included in the touch panel 1976 may detect when the stylus approaches/contacts the operation surface of the touch panel display (similar to the case in which a finger is used for the touch operation).

One or more of the display 1975 and the touch panel 1976 are examples of a touch screen panel display as might be implemented according to the present disclosure.

In certain aspects of the present disclosure, the touch panel 1976 may be disposed adjacent to the display 1975 (e.g., laminated) or may be formed integrally with the display 1975. For simplicity, the present disclosure assumes the touch panel 1976 is formed integrally with the display 1975 and therefore, examples discussed herein may describe touch operations being performed on the surface of the display 1975 rather than the touch panel 1976. However, the skilled artisan will appreciate that this is not limiting.

For simplicity, the present disclosure assumes the touch panel 1976 is a capacitance-type touch panel technology. However, it should be appreciated that aspects of the present disclosure may easily be applied to other touch panel types (e.g., resistance-type touch panels) with alternate structures. In certain aspects of the present disclosure, the touch panel 1976 may include transparent electrode touch sensors arranged in the X-Y direction on the surface of transparent sensor glass.

The touch panel driver may be included in the touch panel 1976 for control processing related to the touch panel 1976, such as scanning control. For example, the touch panel driver may scan each sensor in an electrostatic capacitance transparent electrode pattern in the X-direction and Y-direction and detect the electrostatic capacitance value of each sensor to determine when a touch operation is performed. The touch panel driver may output a coordinate and corresponding electrostatic capacitance value for each sensor. The touch panel driver may also output a sensor identifier that may be mapped to a coordinate on the touch panel display screen. Additionally, the touch panel driver and touch panel sensors may detect when an instruction object, such as a finger is within a predetermined distance from an operation surface of the touch panel display screen. That is, the instruction object does not necessarily need to directly contact the operation surface of the touch panel display screen for touch sensors to detect the instruction object and perform processing described herein. For example, in certain embodiments, the touch panel 1976 may detect a position of a user's finger around an edge of the display panel 1975 (e.g., gripping a protective case that surrounds the display/touch panel). Signals may be transmitted by the touch panel driver, e.g. in response to a detection of a touch operation, in response to a query from another element based on timed data exchange, etc.

The touch panel 1976 and the display 1975 may be surrounded by a protective casing, which may also enclose the other elements included in the user device 1905. In certain embodiments, a position of the user's fingers on the protective casing (but not directly on the surface of the display 1975) may be detected by the touch panel 1976 sensors. Accordingly, the controller 1974 may perform display control processing described herein based on the detected position of the user's fingers gripping the casing. For example, an element in an interface may be moved to a new location within the interface (e.g., closer to one or more of the fingers) based on the detected finger position.

Further, in certain embodiments, the controller 1974 may be configured to detect which hand is holding the user device 19, based on the detected finger position. For example, the touch panel 1976 sensors may detect a plurality of fingers on the left side of the user device 1905 (e.g., on an edge of the display 1975 or on the protective casing), and detect a single finger on the right side of the user device 1905. In this scenario, the controller 1974 may determine that the user is holding the user device 1905 with his/her right hand because the detected grip pattern corresponds to an expected pattern when the user device 1905 is held only with the right hand.

The operation key 1977 may include one or more buttons or similar external control elements, which may generate an operation signal based on a detected input by the user. In addition to outputs from the touch panel 1976, these operation signals may be supplied to the controller 1974 for performing related processing and control. In certain aspects of the present disclosure, the processing and/or functions associated with external buttons and the like may be performed by the controller 1974 in response to an input operation on the touch panel 1976 display screen rather than the external button, key, etc. In this way, external buttons on the user device 1905 may be eliminated in lieu of performing inputs via touch operations, thereby improving water-tightness.

The antenna 2070 may transmit/receive electromagnetic wave signals to/from other external apparatuses, and the short-distance wireless communication processor 1971 may control the wireless communication performed between the other external apparatuses. Bluetooth, IEEE 802.11, and near-field communication (NFC) are non-limiting examples of wireless communication protocols that may be used for inter-device communication via the short-distance wireless communication processor 1971.

The user device 1905 may include a motion sensor 1972. The motion sensor 1972 may detect features of motion (i.e., one or more movements) of the user device 1905. For example, the motion sensor 1972 may include an accelerometer to detect acceleration, a gyroscope to detect angular velocity, a geomagnetic sensor to detect direction, a geo-location sensor to detect location, etc., or a combination thereof to detect motion of the user device 1905. In certain embodiments, the motion sensor 1972 may generate a detection signal that includes data representing the detected motion. For example, the motion sensor 1972 may determine a number of distinct movements in a motion (e.g., from start of the series of movements to the stop, within a predetermined time interval, etc.), a number of physical shocks on the user device 1905 (e.g., a jarring, hitting, etc., of the electronic device), a speed and/or acceleration of the motion (instantaneous and/or temporal), or other motion features. The detected motion features may be included in the generated detection signal. The detection signal may be transmitted, e.g., to the controller 1974, whereby further processing may be performed based on data included in the detection signal. The motion sensor 1972 can work in conjunction with a Global Positioning System (GPS) section 1979. The GPS section 1979 detects the present position of the user device 1905. The information of the present position detected by the GPS section 1979 is transmitted to the controller 1974. An antenna 1980 is connected to the GPS section 1979 for receiving and transmitting signals to and from a GPS satellite.

The user device 1905 may include a camera section 1973, which includes a lens and shutter for capturing photographs of the surroundings around the user device 1905. In an embodiment, the camera section 1973 captures surroundings of an opposite side of the user device 1905 from the user. The images of the captured photographs can be displayed on the display 1975. A memory section saves the captured photographs. The memory section may reside within the camera section 1973 or it may be part of the memory 1978. The camera section 1973 can be a separate feature attached to the user device 1905 or it can be a built-in camera feature. According to an embodiment, the camera section 1973 of the user device 1905 can be implemented in order to acquire a single image or a series of images of anatomy of a patient. For instance, the camera section 1973 of the user device 1905 can be used to capture a single image or a series of images of a head of a patient.

Further to the above, the camera section 1973 of the user device 1905 can include both 2D and 3D capacities. In an embodiment, the memory 1978 can store instructions for executing the method of the present disclosure via a user interface of a software application. The user interface can be displayed via the touch panel 1976, the touch panel 1876 being formed integrally with the display 1975. The method of the present disclosure can be by performed responsive to user interaction with the user device 1905 via the user interface, the user interface being controlled by a processor executing the software application displayed on the touch panel 1976. In an embodiment, the memory 1978 can be a remote server in communication with the user device 1905 via the wireless communication processor 1966. Similar to the memory 1978 local to the user device 1905, the remote server can store instructions for executing the software application and any of the processes described herein.

According to an embodiment, each of the above-described processing sections can be a central processing unit such as a Xenon or Core processor from Intel of America or an Opteron processor from AMD of America, or may be other processor types that would be recognized by one of ordinary skill in the art. In one embodiment, the processing sections can be implemented on an FPGA, ASIC, PLD or using discrete logic circuits, as one of ordinary skill in the art would recognize. Further, the processing sections may be implemented as multiple processors cooperatively working in parallel to perform the instructions of the processes described above.

Obviously, numerous modifications and variations are possible in light of the above teachings. It is therefore to be understood that within the scope of the appended claims, the disclosure may be practiced otherwise than as specifically described herein. As will be understood by those skilled in the art, the systems and methods of the present disclosure may be embodied in other specific forms without departing from the spirit or essential characteristics thereof.

1. A system, comprising:
an image sensor configured to acquire a plurality of images of a body part of a patient; and
processing circuitry configured to
receive the plurality of images of the body part of the patient,
isolate a foreground region of interest including the body part of the patient in the plurality of images,
determine image sensor poses in three-dimensional space based on depth data, the depth data including depth values corresponding to pixels of each image of the body part of the patient,
generate a combined point cloud of pixels corresponding to the foreground region of interest based on the depth data and the image sensor poses, and
generate a mesh surface of the body part of the patient based on the combined point cloud.

2. The system according to claim 1, wherein the processing circuitry is further configured to calculate a blur metric for each image and generate a deblurred image for an image having a calculated blur metric exceeding a blur metric threshold.

3. The system according to claim 1, wherein the processing circuitry is further configured to remove an image from the plurality of images when a number of non-zero depth values in the foreground region of interest of the image is less than a depth values threshold.

4. The system according to claim 1, wherein the processing circuitry is further configured to determine whether the plurality of images include a closed loop scan of the body part of the patient.

5. The system according to claim 4, wherein the processing circuitry is configured to identify a presence of matching features in a subset of images of the plurality of images to identify the closed loop scan.

6. The system according to claim 5, wherein the subset of images includes an initial image of the plurality of images and a final image of the plurality of images.

7. The system according to claim 5, wherein the processing circuitry is further configured to correct the image sensor poses based on the matching features in the subset of images.

8. The system according to claim 1, wherein the processing circuitry is further configured to determine a surface coverage of the body part of the patient based on the image sensor poses.

9. The system according to claim 1, wherein the processing circuitry is further configured to determine the image sensor poses using a machine learning model.

10. The system according to claim 1, wherein the processing circuitry is further configured to calculate a blur metric for each image and select an image from the plurality of images for foreground estimation based on the blur metrics.

11. The system according to claim 1, wherein the processing circuitry is further configured to calculate a blur metric and/or a motion metric for at least one image and modify an exposure time of the image sensor and/or an image capture frame rate of the image sensor based on the blur metric and/or the motion metric.

12. The system according to claim 1, wherein the processing circuitry is further configured to generate the mesh surface using a Poisson surface reconstruction, an Alpha shape reconstruction, or a ball pivoting algorithm.

13. The system according to claim 1, further comprising a depth sensor configured to acquire the depth data.

14. The system according to claim 1, wherein the body part is a head of the patient having a cranial shape and the processing circuitry is further configured to identify one or more landmarks on the mesh surface and calculate at least one cranial parameter based on the one or more landmarks, the at least one cranial parameter being one selected from a group including cephalic index and cranial vault asymmetry index.

15. The system according to claim 14, wherein the one or more landmarks include a nasion and a tragion.

16. The system according to claim 14, wherein the processing circuitry is further configured to compare the at least one cranial parameter to a pre-determined threshold of the at least one cranial parameter and determine, based on the comparison, an abnormality of the cranial shape of the head of the patient using a machine learning model.

17. The system according to claim 14, wherein the processing circuitry is further configured to determine a cranial contour based on the mesh surface.

18. The system according to claim 14, wherein the head of the patient is covered by an opaque cap having one or more visual features and the one or more visual features are used to isolate the foreground region.

19. A non-transitory computer-readable storage medium for storing computer readable instructions that, when executed by a computer, cause the computer to perform a method, the method, comprising:
receiving a plurality of images of a body part of a patient and depth data including depth values corresponding to pixels of each image of the body part of the patient;
isolating a foreground region of interest including the body part of the patient in the plurality of images;
determining image sensor poses in three-dimensional space based on the depth data;
generating a combined point cloud of pixels corresponding to the foreground region of interest based on the depth data and the image sensor poses; and
generating a mesh surface of the body part of the patient based on the combined point cloud.

20. A method, comprising:
receiving, via processing circuitry, a plurality of images of a body part of a patient and depth data including depth values corresponding to pixels of each image of the body part of the patient;
isolating, via the processing circuitry, a foreground region of interest including the body part of the patient in the plurality of images;
determining, via the processing circuitry, image sensor poses in three-dimensional space based on the depth data;
generating, via the processing circuitry, a combined point cloud of pixels corresponding to the foreground region of interest based on the depth data and the image sensor poses; and
generating, via the processing circuitry, a mesh surface of the body part of the patient based on the combined point cloud.
